(19) United States
(12) Patent Application Publication
(10) Pub. No.: US 2005/0037452 A1
(43) Pub. Date: Feb. 17, 2005

(54) Biofermentor for the treatment of biologically convertible matter

(76) Inventors: Michel Deblois, Saint- Laurent Ile d'Orleans (CA); Jacques Goulet, Sainte-Famille Ile d'Orleans (CA)

Correspondence Address: OGILVY RENAULT, 1981 MCGILL COLLEGE AVENUE, SUITE 1600, MONTREAL, QC, H3A2Y3, CA

(21) Appl. No.: 10/488,110

(22) PCT Filed: Sep. 10, 2002
(86) PCT No.: PCT/CA02/01404

(30) Foreign Application Priority Data
Sep. 10, 2001 (GB) 0121735.5

(51) Int. Cl. C12P 1/00; C12N 1/18

(52) U.S. Cl. 435/41; 435/254.2

(57) ABSTRACT

The present invention relates to a method and an apparatus for treating biologically convertible matter by contacting same with microorganisms deposited and immobilized on support carriers. The apparatus is made of a bioreactor comprising three sections, namely a distribution chamber, a bioreactor chamber and a collection chamber. The bioreactor chamber comprises the above mentioned support carriers that are resistant against biodegradation. The modular bioreactor does not require mechanical aeration and may operate continuously, therefore reducing operation costs.

### TECHNICAL FIELD

The present invention relates to a bioreactor and a method for treating biologically convertible matter to provide a conversion material. The bioreactor is designed to allow the treatment of a liquid, solid or gaseous biologically convertible matter with microorganisms, such as yeasts, yeast like, bacteria or mixtures thereof. The treated biologically convertible matter results in a material, hereinafter referred to as conversion material, that is biologically compatible, detoxified and/or reusable.

### BACKGROUND ART

With the increase in urbanization, the development of industrial technologies and the advancement of the consuming society, there has been a rapid increase in biologically convertible production. To solve this problem, many approaches, based on the nature of the biologically convertible matter, have been adopted.

One of the most common way to handle domestic garbage is to use a sanitary landfill where organic and inorganic biologically convertible matters are deposited and further recovered with a layer of soil. Although this procedure is the cheapest among biologically convertible management technologies, organic matter is slowly decomposed by anaerobic processes, producing important quantities of methane gas and heavy metals or other ground water hazardous pollutants. As this method also leaves important quantities of inorganic matter in the environment, important efforts have been made to recycle and revalorize products such as plastics, papers and metals, thereby contributing to reduce the amount of garbage dumped in landfill each years.

While an important part of solid waste production is inert, being composed of glass, metals and plastics, several solid or liquid wastes, such as kitchen scraps, sewage sludge or animal waste, can be decomposed. Composting of these organic wastes appears to be an interesting alternative for the treatment of organic waste. Briefly, composting consists in the conversion of organic wastes into an humus like substance that can be used as soil, by the action of a microbial ecosystem. This technique is more complex than landfill disposal since it requires an initial sorting of the solid biologically convertible into organic and inorganic matter.

A common problem observed with the latter biologically convertible management solutions is their incapacity to treat liquid biologically convertible from a domestic or industrial origin. Domestic biologically convertible water is generally disposed in sewers while industrial liquids often need to be processed prior to disposal. Moreover, valorization of liquid biologically convertibles, such as milk plant by-products or animal manure is often very limited because their treatment is not economically profitable or offers limited efficiencies.

Indeed, techniques such as bioconversion of biologically convertible matter using flocculation, decantation, and microbial treatment of biologically convertible water have been investigated. Contaminated sludges often remain present and their valorization is limited to fertilization. Other biochemical processes encourage the use of microorganisms in bioreactor devices to process biologically convertible matters, where bacteria are most commonly used as microbial cells on an organic support, which is often peat. Although these processes appear to be good solutions to pollution problems, they require a lot of energy, thus being quite expensive. As for other technologies based upon an aerobic digestion that have been developed for the production of gas from pig manure, high cost limits the use of such technologies since production costs must be maintained as low as possible.

In addition to requiring significant quantities of energy, these technologies often involve batch processes, while economics and technical considerations generally favor the utilization of a continuous process for the purpose of biologically convertible treatment. With few exceptions, commercial microorganism propagation processes are conducted as batch or semi-batch processes. Continuous processing has proven to be difficult and, indeed, undesirable for many microorganism propagation systems owing to the inability, among others, to achieve the degree of control required in such processes. This is particularly true with respect to control over the concentration of gaseous materials necessary, or desirably present, in a propagation process, such as oxygen in aerobic propagation systems.

Another significant limitation on the possible use of continuous processing in microorganism propagation systems is the substantially universal requirement that the culture medium has to be purified to remove therefrom microorganisms or other materials which might contaminate the desired microorganisms or the products sought to be recovered therefrom. The degree of purification needed is generally quite high and may involve a number of heat treatments, filtrations or other means for removing undesired materials, such as pathogen bacteria, from the culture medium. In such circumstances, it is quite difficult to develop a process which, from medium purification through microorganism propagation, is truly continuous.

Several attempts were made to refine bioreactor technology to improve its economical reliability on the aspects of energy demand, continuous processing as well as valorization of the biologically convertible matter. An important part of these efforts was dedicated to the agricultural industry, more particularly in swine manure treatment. There are many techniques presently in use to dispose of swine biologically convertible products, most of which create some form of pollution. The most commonly used disposal way is the construction of large artificial pounds or pools or the use of natural pounds and disposal of the animal sewage in these pounds, for further spreading it on a lot reserved to such uses. This practice creates large odorous areas that attract flies and mosquitos, in addition to represent severe contamination risks for surrounding water sources.

U.S. Pat. No. 3,846,558 discloses the valorization of swine sewage by processing it into high protein animal food supplement, by submitting it to microorganism bioconversion.

U.S. Pat. No. 5,202,935 discloses a continuous process for treating biologically convertible matter in a bioreactor using aerobic microorganisms. There still remains a problem associated with the energy demand for aeration.

U.S. Pat. No. 4,665,027 discloses a method for preparing a volatile fermentation product from a non-volatile fermentation substrate. The method makes use of immobilized cells in a reactor-separator including a fermenter enriching section and a fermenter stripping section. In operation, a fermenting broth is contacted by both the immobilized cells and a stripping gas phase.

Considering the state of the art described above, it would thus become desirable to provide a method or an apparatus that convert different types of biologically convertible matter into reusable or detoxified material, while showing economical interests in a continuous or batch process.

### DISCLOSURE OF INVENTION

It is an object of the present invention to convert biologically convertible matter into biomass. It is another object of the present invention to provide a method for the extraction of different compounds or components from biologically convertible matter. It is another object of the invention to provide a modular bioreactor that does not require mechanical aeration and operates continuously, therefore reducing operation costs.

Another object of the present invention is to provide a method for treating biologically convertible matter comprising converting the biologically convertible matter into biomass by contacting said biologically convertible matter with yeasts immobilized on a support.

Another object of the present invention is to provide an apparatus for microbiologically treating biologically convertible matter by contacting the biologically convertible matter with yeasts immobilized on a support thereby converting the biologically convertible matter into biomass.

It is another object of the present invention to remove heavy metals from a contaminated medium by the fixing action of yeast. It is another object of the present invention to concentrate heavy metals in a designated location, which can facilitate their further handling or treatment.

It is another object of the present invention to provide a method valorizing biologically convertible matter by producing biomass, such as a yeast biomass that can be utilized for example, as a food source or as a fertilizer. It is another object of the present invention to measure the conversion of biologically convertible matter into biomass by using at least one of protein content, weight; volume, number of cells or total adenylate of microorganisms as reference.

Broadly stated, the invention relates to a method for treating biologically convertible matter, which comprises providing supports having yeast cells in a proliferating state and immobilized thereon, and contacting the biologically convertible matter with the supports under conditions to produce and separate therefrom a conversion material.

Preferably, the invention comprises allowing the yeast cells to fix, absorb, metabolize, alter, convert, digest, or degrade the biologically convertible matter.

Most preferably, the invention comprises allowing the yeast cells to cause fermentation of the biologically convertible matter.

In accordance with a preferred embodiment, at least about 0.1 to 90% of the biologically convertible matter is converted into conversion material.

In accordance with another preferred embodiment, a carbohydrate source, a nitrogen source, a mineral, a salt, or a amino acid are added to the biologically convertible matter prior to contacting same with the yeast cells.

The carbohydrate source is preferably selected from the group consisting of amygdalin, arabinose, cellobiose, esculin, fructose, galactose, glucose, gluconate, lactose, maltose, mannitol, mannose, melezitose, melibiose, raffinose, rhamnose, ribose, salicin, sorbitol, sucrose, trehalose, xylose, and the like.

The nitrogen source is preferably selected from the group consisting of urea, ammonium sulfate, and the like that contain assimilable nitrogen.

The biologically convertible matter that is normally treated by the method according to the invention usually comprises manure, a milk fraction or a milk derivative, a solvent, an organic or an inorganic matter, a processed food, a food by-product, a heavy metal, or an aqueous solution.

The method according to the invention may be carried out in a continuous or a batch process.

Preferably, the biologically convertible matter is contacted with the yeast cells in a bioreactor.

The yeast cells are preferably from a wild type strain or a genetically modified strain of yeast. Usually, at least one strain of yeast is used.

The yeasts are preferably immobilized onto the support with glycoleme.

The conversion material usually comprises a biomass composed of treated biologically convertible matter, yeast cells detached from the supports, or a mixture thereof.

The invention also relates to a bioreactor for treating biologically convertible matter, comprising
an upper distribution chamber, a bioreactor chamber disposed below the distribution chamber, means for feeding the biologically convertible matter to the distribution chamber,
means for transferring the biologically convertible matter from the distribution chamber the bioreactor chamber,
a plurality of supports mounted in the bioreactor chamber having yeast cells deposited and immobilized thereon in a viable state, the supports being arranged to cause the yeast cells to contact biologically convertible matter and convert same into a conversion material,
a collection chamber operatively connected to the bioreactor chamber to collect the conversion material; and
means for withdrawing the conversion material from the collection chamber.

According to a preferred embodiment, the bioreactor according to the invention comprises an air inlet disposed on the collection chamber and arranged to allow air to circulate through the bioreactor.

The biologically convertible matter is usually at least one of a liquid, a solid or a gaseous biologically convertible matter, or a mixture thereof.

In accordance with another preferred embodiment, the distribution chamber comprises an exhaust arranged to allow exit of air or other gases ascending in the bioreactor.

Preferably, the distribution chamber comprises at least one sprinkler connected to the feeding means, the sprinkler being arranged for spreadingly distributing the biologically convertible matter on the support.

The bioreactor according to the invention may also comprise means for injecting the biologically convertible matter in gaseous form into the collection chamber and allowing same to contact the supports therein and exit the reactor through the exhaust.

The supports are normally fixed to an external wall of the bioreactor chamber.

The collection chamber preferably comprises to permit passive or active entry of gas into the bioreactor, for example polluted air, organic gas, inorganic gas, or unpolluted air.

The yeast cells are usually composed of at least one species of yeast cells, and they are preferably immobilized on the supports by artificial or natural processes. For example, the yeast cells are immobilized on the supports with glycoleme.

The supports are normally made of a material that is resistant against biodegradation, for example a polymer or a plastic material.

The supports preferably have a planar shape and are disposed vertically. They may also be disposed obliquely or in any suitable manner, as will be understood by one skilled in the art.

According to the present invention, treated biologically convertible matter can be provided on an industrial or a domestic basis and the biologically convertible matter can be composed of at least one of a liquid, a solid or a gas. Biologically convertible matters can be either organic or inorganic materials and can have already been subjected to any kind of treatment, or simply being raw material.

In accordance with the present invention, yeasts of the wild type or genetically modified strains are grown until a yeast layer is formed onto supports provided in the bioreactor chamber of the bioreactor of the invention, wherein the yeast layer can be composed of yeasts belonging to a same strain or a combination of different strains. To do so, the yeast strains are selected prior to treating biologically convertible matter, inoculated onto a support and allowed to multiply onto this support. Multiplication of yeasts includes submitting the yeasts to environmental conditions that are advantageous to proliferation of the yeasts while not being advantageous to proliferation of other microorganisms such as pathogen bacteria. These environmental conditions include temperature, pH and nutrients as well as oxygen availability.

The yeast layer can be immobilized onto a support provided in a bioreactor by artificial or natural processes. For example, without limiting it thereto, the natural process may include immobilization of yeasts with glycoleme produced by the yeasts themselves. The support is preferably made of a material that is resistant against biodegradation, such as, for example, a polymer or a plastic. In addition, this material is preferably made of a washable material. The support can have different configurations or arrangements. It can be configured as honey bee combs, as a mesh, or as a planar shape member. The supports can be disposed horizontally, vertically or diagonally into the fermentation chamber depending if the biologically convertible matter to be treated is solid, liquid, gaseous or a mixture thereof. The supports can be disposed to extend inwardly from the external wall in the bioreactor chamber, alone or in combination with other supports.

In accordance with the present invention, the apparatus has mainly the form of a long vertical cylinder-forming a column, mainly composed of a top distribution chamber, a middle bioreactor or fermentation chamber and a bottom collection section. The distribution chamber allows an optimized falling of the biologically convertible matter onto the supports, whereas the collection chamber allows air circulation into the biofermentor and collects biologically convertible matter or yeasts that fall down from the bioreactor chamber. Air is provided passively or mechanically. The air circulation can be performed only by the ascending movement of warm air in the bioreactor.

The bioreactor or fermentation chamber comprises one or a plurality of supports, that carry immobilized yeasts. The supports are arranged into the bioreactor chamber to allow free circulation of at least one of a solid, a liquid or a gas, wherein the gas includes air.

For the purpose of the present invention the following terms are defined below.

The term “laminar flow” as used herein is intended to mean a placid flow or a flow that is not turbulent.

The term “biomass” as used herein is intended to mean a mass of living or biological material present in a specified area.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 is a longitudinal cross-section view of a modular bioreactor according to the invention, including a distribution chamber, a bireactor chamber, and a collection chamber;

FIG. 2 is a longitudinal cross-section view of a distribution chamber having a direct supply configuration;

FIG. 3 is a longitudinal cross-section view of a distribution chamber having an immersed supply configuration;

FIGS. 4A to 4C are partial perspective views of yeast cell supports, respectively made from a plain wrought material, a mesh type material, and an open-cell foam type material; and

FIG. 5 is a longitudinal cross-section view of a collection chamber having raised outer walls for inserting the bireactor chamber therein.

### MODES OF CARRYING OUT THE INVENTION

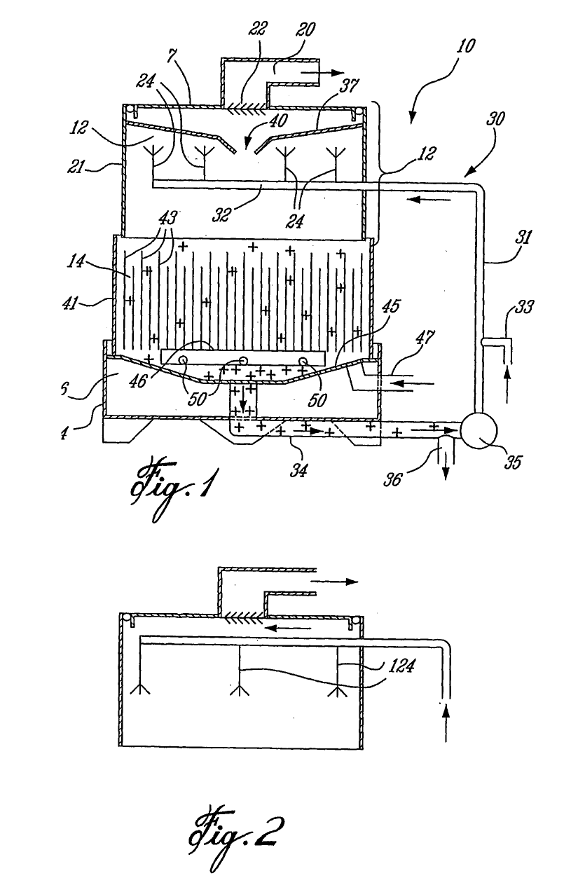

Referring to the drawings, more particularly FIG. 1, it will be seen that an apparatus according to the present invention is in the form of a preferably cylindrical modular bioreactor 10, comprising an upper distribution chamber 12, a middle bioreactor chamber 14 and a lower collection chamber 16. Bioreactor 10 also comprises a piping system for feeding biologically convertible matter therein and exiting conversion material therefrom. To achieve this, there is provided a feeding duct 30, having a first portion 31 mounted outside bioreactor 10, as shown, and a second portion 32, mounted horizontally inside upper distribution chamber 12, also as shown in FIG. 1. To feed biologically convertible matter to feeding duct 30, there is provided a biologically convertible matter inlet 33.

The piping system also comprises an exit duct 34 that terminates at one end in collection chamber 16 and is connected at the other end to a pump 35, that ensures circulation of biologically convertible matter and conversion material throughout the bioreactor, and may also be used for recycling unconverted biologically convertible matter, as will be discussed later. Just short of pump 35, it will be noted that a recovery outlet 36 is provided to recover substantially all the conversion material collected in collection chamber 16, and that is discharged through exit duct 34, where it finally exits from bioreactor 10 via recovery outlet 36. Unconverted biologically convertible matter traveling through exit duct 34 will be separated from the conversion material by any manner known to those skilled in the art and will be directed toward pump 35 for recycling back to distribution chamber 12 via duct portion 31.

Upper distribution chamber 12 is constructed to allow for a uniform and optimized dispensing of the biologically convertible matter to be processed, into bioreactor chamber 14. For this purpose, duct portion 32 exends nearly the entire width of distribution chamber 12 and is connected in known manner to a plurality of sprinklers 24 of known construction, here four, which upwardly mounted in known manner inside distribution chamber 12 and are arranged to project and scatter biologically convertible matter particles or drops, or a gaseous mist against conical baffle 37. Sprinklers 24 are of course adapted to the nature of the biologically convertible matter to be treated and the design of bioreactor chamber 14. Depending whether the biologically convertible matter is a gas, a liquid, a solid or a mixture thereof, sprinklers 24 are preferably constructed to maintain a laminar flow within bioreactor chamber 14. As shown, conical baffle 37 consists of a partition that downwardly extends from walls 21 of distribution chamber 12, and is provided with an opening 40, whose purpose will be discussed later.

Upper distribution chamber 12 will also be seen to comprise an outer wall 21, and a top partition 7 to which an exhaust conduit 20 is connected. The latter is finally provided with a damper drip system 22 of known construction, that is used to control the volume of air or gas that may circulate through bioreactor 10 and capture most of the mist of biologically convertible solution that may be present in the exiting air as will be discussed more in detail later.

Finally, it will be realised that distribution chamber 12 is opened at the bottom to be in full communication with bioreactor chamber 14 as well illustrated in FIG. 1.

As mentioned above, and as shown in FIG. 1, bioreactor 10 also comprises a middle bioreactor chamber 14. The latter is also preferably cylindrical and, as illustrated, it is opened at top and bottom for the purpose of receiving on the one hand the particles, drops or mist that are discharged from distribution chamber 12, and on the other hand, for directly delivering conversion material to collection chamber 16. It will also be noted that bioreactor chamber 14 is wider than distribution chamber 12, so as to prevent particles, drops or mist from falling outside bioreactor chamber 14, being transferred from distribution chamber 12. At the same time, bioreactor chamber 14 is narrower than collection chamber 10, for the same reason, i.e. to prevent any conversion material from being discharged outside collection chamber 16. Of course, any other arrangement known to those skilled in the art may be used as long as no material escapes from bioreactor 10.

In the embodiment illustrated in FIG. 1, bioreactor chamber 14 comprises a cylindrical wall 41 that is opened at both ends to constitute a cylindrical container defining a column into which there is provided a side by side arrangement of spaced apart vertically mounted supports 43 having yeast cells not shown in proliferating state deposited and immobilized thereon. The column is filled with supports 43, each preferably having a thickness between about 0.01 mm and 5 mm, although this value may vary as is well known to those skilled in the art. Supports 43 are vertically packed into bioreactor chamber 14 so as to allow a free circulation of either solid or liquid particles, or drops and/or gases such as air therebetween. The distance between support carriers may be chosen at will by the man of the art, however in practice, a distance between 2 and 15 mm has been found acceptable. It is of course understood that this distance range may vary substantially therefrom.

Referring again to FIG. 1, as mentioned above, bioreactor 10 also comprises a collection chamber 16. The latter is also cylindrical, and and as mentioned above it is wider than bioreactor chamber 14 to receive the bottom portion of bioreactor chamber 14 between its outer walls 44. More particularly, collection chamber 16 is in the form of a cylindrical vat having a top partition in the shape of an Inverted truncated conical member 45. Of course, any other suitable shape for this partition may be adopted as will be understood by one skilled in the art. Conversion material recovery duct 34, mentioned above, opens into top partition 45 as shown to discharge a mixture of biologically convertible matter and conversion material produced in bioreactor chamber 14 which is sent towards outlet 36. As discussed above, the conversion material is recovered through outlet 36 while the remaining biologically convertible matter is recycled towards distribution chamber via pump 35 and ducts 31 and 32.

Normally, it is desirable to introduce air into bioreactor 10 for the purpose of providing aerobic conditions, or merely to ventilate the bioreactor. This is made possible by providing a horizontally mounted perforated duct 46 that Is connected to a gas intake 47 as shown. Air can thus be passively or forceably introduced into bioreactor chamber 14.

On the other hand, if it is intended to treat only biologically convertible gaseous matter, the latter may be introduced via intake 47, where it will travel through bioreactor 14 to be treated therein and through distribution chamber 12 to exit via outlet 20 as a non polluant gas.

Referring to FIG. 2, it will be seen that sprinklers 24 are downwardly oriented for a direct feeding and distribution of the biologically convertible matter to bioreactor chamber 14. In this case, it will be noted that baffle 37 is not present.

The sprinklers are adapted to the nature of the biologically convertible matter that requires a treatment and to the configuration of the bioreactor chamber 14. Depending on whether the biologically convertible matter is a gas, a liquid, a solid or a combination thereof, the sprinklers are designed to maintain a laminar flow within bioreactor chamber 14. A distribution chamber 12 having a direct configuration, i.e. in which the sprinklers directly discharge biologically convertible matter onto the supports as in the embodiments illustrated in FIG. 2, can be used for the treatment of gas and solid/liquid biologically convertible matter. Using this configuration, a biologically convertible matter solution can be directly sprayed onto supports 43 of bioreactor chamber 14. The sprinklers are of course designed for an even distribution of the biologically convertible matter into bioreactor chamber 14.

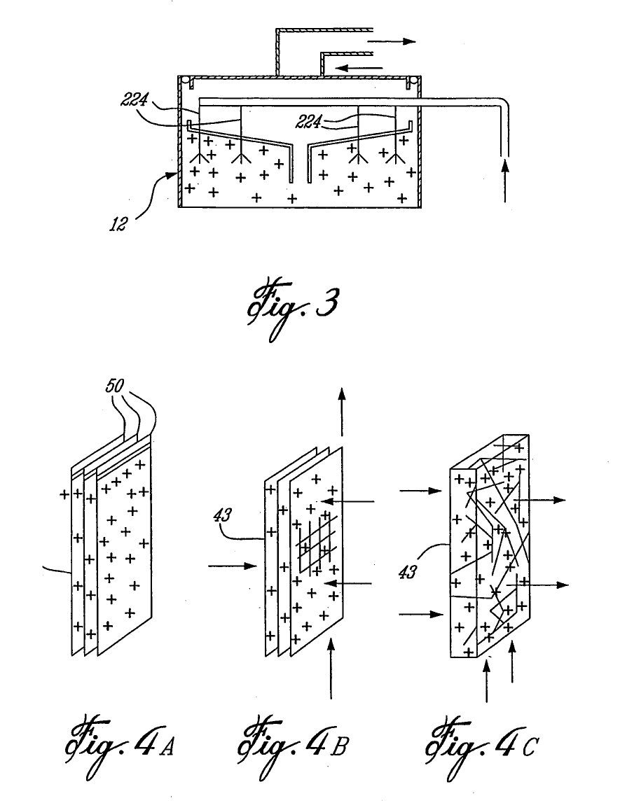

Referring to FIG. 3, it this embodiment is adapted for directly contact of the biologically convertible matter with yeasts when a biologically convertible matter solution is use. In this case, sprinklers 24 are downwardly oriented and are completely immersed below baffle 37 in the biologically convertible matter solution that is submitted to a conversion treatment; in this manner they can induce a cyclonic movement around conical wall 37, within distribution chamber 12. This configuration favors the concentration of recycled yeast from the side, toward the middle of bioreactor chamber 14 down to collection chamber 16 and is particularly used for the treatment of biologically convertible contaminated with, for example but not limited to, heavy metals.

The yeasts may be of one strain or of a pre-selected mixture of yeast strains. Supports 43 preferably have a planar structure and are preferably disposed vertically or obliquely within bioreactor chamber 14. Preferably, multiple supports 43 can be grouped in a bioreactor chamber 14 to maximize the surface of treatment. The distance between supports 43 in a multiple configuration can be variable but must be sufficient to allow circulation of gas, liquid and/or solid. Preferably, the distance between two supports 43 varies between 3.0 mm and 13.0 mm, especially when the biologically convertible matter comprises liquids and solids. Moreover, the space separating two yeast supports 43 must be sufficient to allow a free falling down of yeast excess into collection chamber 16 and fresh air to circulate upwardly within bioreactor chamber 14. The thickness of the supports may also vary, but a thickness of 0.001 mm to 5 mm is preferred to allow for a free circulation of both air and liquid between the plates while maximizing treatment surface.

Supports 43 are made of any suitable material allowing a microorganism to grow as a layer on its surface. The material may either be hydrophilic or hydrophobic, such as loosely woven insect protection screens, geotextiles, sunscreens, and the like. It may also consist of a non woven material with any smooth or grooved surface. However, a washable material is preferred so that the plates may be reusable as opposed to a non reusable material. While supports 43 can be made from a number of materials, a material that is biodegradation resistant is preferred, such as a polymer or a plastic. Other supports, such as peat, are more susceptible to biodegradation. PVC, PP, PE, wood, metal, or the like supports may be used depending on the nature of the yeast cells used for the treatment of biologically convertible matter.

In addition to the chemical composition of supports 43, their chosen structure is based on the composition of the biologically convertible matter to be treated, to insure a maximum exchange of oxygen and to maintain a laminar flow. The choice is entirely left to one skilled in the art.

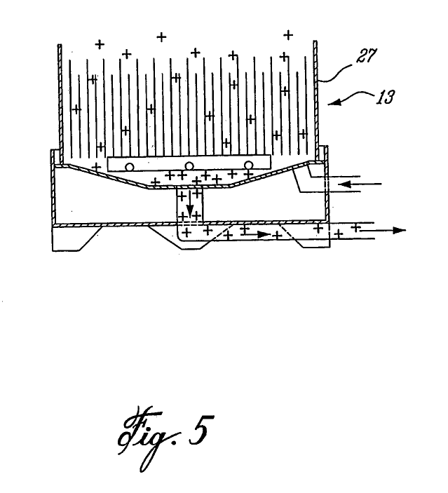

Referring now to FIGS. 4A, 4B and 4C, supports 43 appears as a plate with a solid, tight meshed or porous surface. A solid surface support is preferably used for the treatment of heavy metal-contaminated biologically convertibles, whereas a support made from a tight mesh material is appropriate for the treatment of liquids and solids.

A porous support 43, made from an opened cell foam structure with a variable density and distribution of holes, is however preferred for the treatment of gaseous pollutants. In addition, supports 43 are preferably shaped with an acute upper edge 50, in a manner to ensure an appropriate diffusion of biologically convertible matter when its viscosity in very low.

To immobilize yeast onto support 43 as defined herein, it is inoculated and allowed to multiply onto the support prior to being contacted with biologically convertible matter. The yeast inoculated onto a support can be of a strain selected for a specific biologically convertible matter to be treated prior to inoculation onto the support. The selected yeast can belong to a same strain or a combination of multiple strains, when the latter are wild type strains or genetically modified strains. Allowing the multiplication of yeast includes submitting the yeast to environmental conditions that are advantageous to proliferation of the yeasts while not being advantageous to proliferation of other microorganisms, wherein said environmental conditions include temperature, pH, nutrients and oxygen availability. The yeasts develop until they form a layer, while growth of other microorganisms, including pathogen bacteria, is prevented.

Yeast is immobilized onto fixed support by artificial or natural processes, wherein the natural process is immobilization with yeast-secreted glycoleme.

Treated and rejected biologically convertible matter and detached yeast can be used as soil or food supplement.

Bioreactor 10 is preferably in the form of a column to facilitate a natural ventilation or oxygenation of bioreactor chamber 14. As yeast bioconversion of biologically convertible matter is an exothermic process, the surrounding air within bioreactor chamber 14 is warmed and is naturally evacuated via exhaust conduit 20 located at the top of the distribution chamber 12, therefore creating a low negative pressure zone within the bioreactor. Fresh air is passively introduced in the bioreactor via collection chamber 16. Perforated air duct 46 introduces the air required for the aerobic treatment of biologically convertible matter and to necessary regulate the temperature of bioreactor chamber 14. Moreover, perforated air duct 46 can be used to inject a pollutant gas concomitantly to air. The air is adequately diffused into bioreactor chamber 12 through openings 50 provided in perforated air duct 46. Duct 34 allows the recycling of yeasts that fall down into collection chamber 16 and recirculation of the biologically convertible matter that is not processed during its passage in bioreactor chamber 14. During the treatment, yeast activity warms the air that is later on evacuated, thereby causing an increased funnel effect and by consequence, an increase of oxygen availability. An increase of air circulation within bioreactor chamber 14, as a result of the funnel effect, cools down bioreactor 10, allowing the maintenance of an optimal yeast activity without external control. As the air required for the digestion process naturally circulates within the bioreactor, less mechanical aeration is needed, therefore reducing the energy demand for aeration. Air, alone or in combination with other gases can be mechanically forced into the bioreacxtor, by any known means. Damper drip system 22, located at the top of distribution chamber 12 controls the exit of air from the bioreactor through exhaust conduit 20 and captures most of the residual mist produced by the biologically convertible solution contained in the exhaust air.

The amount of air needed for the aerobic process may enter freely or be forced into bioreactor 10 via collection chamber 16 where it circulates counter currently in upward direction. The free circulation of the air and of a thin film of nutrition liquid media favors the diffusion of the nutrient and dissolution of the oxygen that are required for the conversion of biologically convertible matter into biomass by yeast cells. Furthermore, the presence of multilayer supports considerably increases the reaction surface in the bioreactor which on the other hand allows to reduce the size of the column. The end product, such as a protein, resulting from biotransformation by yeast cells, is collected at the bottom of the column through duct 34, thereby allowing a continuous as well as a batch process to be performed, involving the treatment of biologically convertible matter. Pump 35 allows the unconverted biologically convertibler matter and yeasts that are dumped into collection chamber 16 to be re-introduced at the top of bioreactor chamber 12.

The present invention enables to treat a wide variety of biologically convertible matters. The invention can be used for the treatment of a gas, a liquid, a solid or a combination thereof. In addition to the physical state of the biologically convertible matter, the chemical nature can vary, for example, the biologically convertible matter can be organic, inorganic or both; it can also be of an industrial or a domestic nature. The present invention provides an effective route for treating biologically convertible derived from food factories, cheese factories, slaughter houses, etc, by-products obtained in the production of animals, such as pigs, bovines, chickens, and the like, as well as in the production of vegetables, in non-alimentary industries such as in the pulp and paper industry, the card board industry, and the like, and in plants that produce harmful gases including styrene, xylene, and the like, as well as biologically convertible of domestic origin originating from septic tanks, purification factories, and the like. Biologically convertible matter can be a raw material or a material that has been submitted to any treatment prior to being converted into biomass using the method described herein. For example, liquid and solid phases can be separated, where the liquid phase can be treated using the method described herein while the solid phase is directly used as fertilizer. A complex liquid biologically convertible matter that liberates gases can be used as nutrient source for yeasts wherein the gases are introduced concomitantly with the air required for the aerobic process within the bioreactor. In addition, a biologically convertible matter that has already been subjected to treatment using the method according to the invention can be recycled to modular bioreactor 10 by reentering distribution chamber 12 through piping system 30.

While the treated biologically convertible solution recovered in collection chamber 16 maintains an oxygen concentration of at least 0.25 ppm, and untreated biologically convertible matter and nutrients are introduced into piping system 30 and mixed with recycled treated biologically convertible solution, an equivalent volume of treated biologically convertible solution is drained off at outlet 36. The continuous process in bioreactor 10 thus allows for the production of a biomass in bioreactor chamber 14 and its recovery at the base thereof. This characteristic permits a continuous operation of bioreactor 10 thus limiting the investment required for a batch treatment and ensuring a constant control of the system. The continuous process in the bioreactor operates with a minimum of energy, since pump 35 and, In some cases, a ventilator (not shown) are the only parts that require energy sources, therefore reducing the cost of biologically convertible treatment. Alternatively, bioreactor 10 can be used to treat biologically convertible matter in a batch process.

The versatility of the bioreactor is ensured by allowing modifications to distribution chamber 12, bioreactor chamber 14 and collection chamber 16 that can independently be adapted to the biologically convertible matter that has to be treated. Alternatively, the various sections or chambers of the bioreactor of the instant invention can be sealed so as to form a monobloc like bioreactor, therefore preventing liquid or air interchange between the interior and the outside environment of the bioreactor. It will be recognized by someone skilled in the art that the bioreactor of the present invention can be produced as a one piece module comprising the three chambers described above.

According to the present invention, biologically convertible matter may be contacted with a yeast layer with or without complementary nutrients, that may include a carbon source, a nitrogen source, minerals, salts and amino acids.

The conversion of biologically convertible matter into biomass is initiated by contacting the biologically convertible matter with yeasts, and this normally occurs mechanically

Several treatment cells can be fed by the same distribution chamber. This modular approach allows an easy adjustment of the bioreactor capacity to the volume of the material to be treated and to various treatment scenarios, e.g. alteration of cells during treatment and those that are at rest. It is also possible to stock auxiliary cells for long periods many months before using them.

The present invention will be more readily understood by referring to the following examples which are given to illustrate the invention and without limitation.

### EXAMPLE I

### Treatment of Pig Manure

Pig manure is injected at the top of a bioreactor according to the invention, which has an indirect spray configuration since pig manure comprises solids, liquids and gases that require misting within air for an optimal bioconversion reaction. As pig manure is rich in ammonia, the treatment of liquid containing yeast must be supplemented with a source of carbon such as molasses and supplied with continuous oxygen. The bioreactor can alternatively be aerated at the bottom with polluted air originating from a pig sty, which contains ammonia.

The selection of the yeast or other microorganisms to be immobilized on the supports, is made according to the capacity of the species to fix and to transform if necessary the substrate to be treated. The supports used in the bioreactor chamber are perforated polyester or vinyl membranes having a plurality of orifices varying from 1.6 to 6.5 mm in diameter, where each support is maintained apart from each other at a maximum distance of 1.3 mm. Alternately, the supports used for the treatment of a substrate such as pig manure may comprise glass and/or polyester fiber opened lattices covered with PVC with openings of 1.6 mm in diameter, such support permitting fixation of yeast and exchange of oxygen; the lattice is maintained vertically by way of a mechanical support; a framing maintains each support flat and vertical; and the frames are wrapped together with polymer panels. The result is the formation of a biomass from pig manure and a substantial decrease of the odors resulting from a removal of ammonia by the yeast.

The ammonia content in the liquid and the solid fractions was reduced to less than 10% of the initial content. The phosphorus content in the liquid content was reduced in the same proportion. Most of the phosphorus was concentrated in the solid fraction.

### EXAMPLE II

### Treatment of Cheese Whey

A cheese whey containing about 4%-5% of lactose is mixed with a solution containing 40% urea by a static mixer and injected at the top of a bioreactor as described in example 1. The bioreactor is aerated with ambient air. The yeasts selected and immobilized in the bioreactor can assimilate the lactose carbon source, and the nitrogen of the urea to produce a yeast biomass. The solution recovered at the bottom of the bioreactor has a lactose content lower than 0.1%, a pH under 4.5 and contains approximately 2%-3% of organic matter (yeasts biomass).

The solution recovered at the bottom is centrifuged. Most of the yeast is concentrated at a level of 15%-18%. The protein content of the solid fraction, the biomass, is close to 56% (dry basis). The pH of the liquid fraction is raised to 6 by adding lime. The chemical oxygen demand of the residual liquid fraction is reduced to less than 15% of the initial cheese whey material.

### EXAMPLE III

### Treatment of Brewery Waste Water

The waste water recovered from the first rinse of a beer storage tank is mixed by a static mixer with a sucrose solution and injected at the top of a bioreactor as in examples 1 and 2. The waste water contains the wort fraction and the microorganisms immobilized in the bioreactor are yeasts. The air enters the bioreactor by the bottom section, in a countercurrent flow fashion.

A biomass is produced and recovered at the bottom of the bioreactor. The biomass is centrifuged and removed from the liquid fraction. The chemical oxygen demand of the residual liquid fraction is reduced at less than 40% of the initial waste water material

### EXAMPLE IV

### Treatment of Air Containing Xylene

The air containing xylene from mirror manufacture is washed by a trickling aqueous solution to trap xylene in a liquid material. The aqueous solution containing xylene is mixed with a urea solution by a static mixer and injected at the top of the bioreactor. The yeast culture selected fix the xylene. The air discharged outside of the plant had a xylene concentration reduced by 60% of its initial content.

Finally, it is understood that two or more biologically convertible matters may by treated simultaneously in the same bioreactor.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for treating biologically convertible matter, which comprises providing non biodegradable supports having microorganisms in a biologically active condition immobilized thereon, disposing said supports in an arrangement to enable free circulation of a gas through said arrangement, and contacting said biologically convertible matter with said supports under conditions to produce and separate therefrom a conversion material, or to concentrate and fix elements of said biologically convertible material.

2. The method of claim 1, wherein said microorganisms comprise yeast, yeast like, bacteria or mixtures thereof.

3. The method of claim 1, wherein said microorganisms comprise yeast cells.

4. The method of claim 1, which comprises allowing said microorganisms to fix, absorb, metabolize, alter, convert, digest, or degrade said biologically convertible matter.

5. The method of claim 1, which comprises allowing said microorganisms to cause fermentation of said biologically convertible matter.

6. The method of claim 3, which comprises converting at least about 0.1 to 90% of said biologically convertible matter into said conversion material.

7. The method of claim 1, which comprises concentrating elements present in said biologically convertible matter, and fixing and recovering same.

8. The method of claim 1, which comprises adding a carbon source, a nitrogen source, a mineral, a salt, an amino acid or a product containing same to said biologically convertible matter prior to contacting it with said microorganisms

9. The method of claim 8, wherein said carbohydrate source is selected from the group consisting of starch, amygdalin, arabinose, cellobiose, esculin, fructose, galactose, glucose, gluconate, lactose, maltose, mannitol, mannose, melezitose, melibiose, raffinose, rhamnose, ribose, salicin, sorbitol, sucrose, trehalose, xylose, cellulose, and organic acids or mixtures thereof products containing same.

10. The method of claim 1, wherein said biologically convertible matter is selected from the group consisting of manure, a milk fraction or a milk derivative, a solvent, an organic or an Inorganic matter, a processed food, a food by-product, a heavy metal, and an aqueous solution.

11. The method of claim 1, which comprises treating said biologically convertible matter in a continuous or a batch process.

12. The method of claim 1, which comprises contacting said biologically convertible matter with said microorganisms in a bioreactor.

13. The method of claim 1, wherein microorganisms are from a wild type strain or a genetically modified strain of yeast.

14. The method of claim 1, wherein said microorganisms comprises at least one strain of yeast.

15. The method of claim 1, wherein said microorganisms are immobilized onto said support under conditions promoting formation of glycoleme.

16. The method of claim 1, wherein said microorganisms are immobilized onto said supports by means of a thixotrope.

17. The method of claim 1, wherein said conversion material comprises a biomass composed of treated biologically convertible matter, microorganisms detached from said supports, or a mixture thereof.

18. A bioreactor for treating biologically convertible matter, comprising
an upper distribution chamber,
a bioreactor chamber disposed below said distribution chamber,
means for feeding said biologically convertible matter to said distribution chamber,
means for discharging said biologically convertible matter from said distribution chamber into said bioreactor chamber,
a plurality of non biodegradable supports mounted in said bioreactor chamber having microorganisms in a biologically active condition immobilized thereon, said supports being arranged to cause said microorganisms to contact said biologically convertible matter and convert same into a conversion material, or concentrate and fix elements of said biologically convertible matter,
a collection chamber operatively connected to said bioreactor chamber to collect said conversion material or recover said fixed elements; and
means for withdrawing said conversion material or fixed elements from said collection chamber.

19. The bioreactor of claim 18, which comprises an air inlet disposed on said collection chamber and arrange to allow air to circulate through said bioreactor.

20. The bioreactor of claim 18, wherein said biologically convertible matter is at least one of a liquid, a solid or a gaseous biologically convertible matter.

21. The bioreactor of claim 19, wherein said distribution chamber comprises an exhaust arranged to allow exit of air ascending in said bioreactor.

22. The bioreactor of claim 18, wherein said distribution chamber comprises at least one sprinkler connected to said feeding means for spreadingly distributing said biologically convertible matter on said support.

23. The bioreactor of claim 18, which comprises means for injecting said biologically convertible matter in gaseous form into said collection chamber and allowing same to rise and contact said supports therein and exit said reactor through said exhaust.

24. The bioreactor of claim 18, wherein said supports are fixed to an external wall of the bioreactor chamber.

25. The bioreactor of claim 24, wherein said supports are horizontally, vertically, diagonally or spirally arranged.

26. The bioreactor of claim 18, wherein said collection chamber comprises means to permit passive or active entry of gas Into said bioreactor.

27. The bioreactor of claim 26, wherein said gas is polluted air, organic gas, inorganic gas, or unpolluted air.

28. The bioreactor of claim 18, wherein said microorganisms comprise yeasts, yeast like, bacteria or mixtures thereof.

29. The bioreactor of claim 28, wherein said microorganisms comprise yeast cells.

30. The bioreactor of claim 29, wherein said yeast cells are composed of at least one species of yeast cells.

31. The bioreactor of claim 228, wherein said microorganisms are immobilized on said supports by artificial or natural processes.

32. The bioreactor of claim 29, wherein said yeast cells are immobilized on said supports under conditions promoting formation of glycoleme.

33. The bioreactor of claim 29, wherein said yeast cells are immobilized on said supports by means of a thixotrope.

34. The bioreactor according to claim 18, wherein said supports are made of a polymer.

35. The bioreactor of claim 18, wherein said supports have a planar shape and are disposed vertically.
